(19) United States
(12) Patent Application Publication
(10) Pub. No.: US 2006/0040312 A1
(43) Pub. Date: Feb. 23, 2006

(54) Uses of kappa-conotoxin PVIIA

(75) Inventors: Ann H. Cornell-Bell, Westbrook, CT (US); Karen E. Pemberton, Guilford, CT (US); Davis L. Temple JR., Clinton, CT (US); Richard T. Layer, Sandy, UT (US); R. Tyler McCabe, Salt Lake City, UT (US); Robert M. Jones, San Diego, CA (US)

Correspondence Address: ROTHWELL, FIGG, ERNST & MANBECK, P.C., 1425 K STREET, N.W., SUITE 800, WASHINGTON, DC, 20005, US

(73) Assignee: Cognetix, Inc., Salt Lake City, UT (US)

(21) Appl. No.: 11/259,039

(22) Filed: Oct. 27, 2005

Related U.S. Application Data
(63) Continuation of application No. 10/627,685, filed on Jul. 28, 2003, now abandoned.
Application No. 10/627,685 is a continuation of application No. 09/666,837, filed on Sep. 21, 2000, now abandoned.
(60) Provisional application No. 60/219,438, filed on Jul. 20, 2000.
(60) Provisional application No. 60/155,135, filed on Sep. 22, 1999.

(51) Int. Cl. C12Q 1/68; A61K 38/16

(52) U.S. Cl. 435/6; 514/7; 514/12

(57) ABSTRACT

The invention relates to uses of kappa-conotoxin PVIIA (κ-PVIIA), analogs and derivatives for activating ATP-sensitive K⁺ channels. The activation of ATP-sensitive K⁺ channels is useful for opening K_{ATP} channels which can be used to treat a wide range of disease and injury states, including cerebral and cardiac ischemia and asthma.

### CROSS-REFERENCE TO RELATED APPLICATIONS

The present application is a continuation of U.S. patent application Ser. No. 10/627,685, filed on 28 Jul. 2003, which is a continuation of U.S. patent application Ser. No. 09/666,837, filed on 21 Sep. 2000. Ser. No. 09/666,837 claims benefit under 35 U.S.C. § 119(e) to U.S. provisional patent application No. 60/219,438, filed on 20 Jul. 2000, and to U.S. provisional patent application No. 60/155,135, filed on 22 Sep. 1999. Each application is incorporated herein in its entirety by reference.

### BACKGROUND OF THE INVENTION

The invention relates to uses of kappa-conotoxin PVIIA (κ-PVIIA), analogs and derivatives for activating (i.e., opening) ATP-sensitive K⁺ channels. The activation of ATP-sensitive K⁺ channels is useful for treating many physiological disorders, as described in further detail herein.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

κ-PVIIA, a 27 amino acid peptide that was originally purified from the venom of the purple cone snail Conus purpurascens (Terlau et al., 1996; U.S. Pat. No. 5,672,682) has been previously identified as a potent antagonist of the Shaker H4 potassium channel (IC₅₀˜60 nM). In the same study, no detectable activity on the voltage-gated potassium channels Kv1.1 or Kv1.4 (Terlau et al., 1996) was noted. Chimeras constructed from the Shaker and the Kv1.1 K⁺ channels have identified the putative pore-forming region between the fifth and sixth transmembrane region as the site of the toxin sensitivity (Shon et al., 1998). It appears that κ-PVIIA interacts with the external tetraethyl-ammonium binding site on the Shaker channel. Although both κ-PVIIA and charybdotoxin inhibit the Shaker channel, they must interact differently. The F425G Shaker mutation increases charybdotoxin affinity by three orders of magnitude but abolishes κ-PVIIA sensitivity (Shon et al., 1998). κ-PVIIA appears to block the ion pore with a 1:1 stoichiometry, and its binding to open or closed channels is very different (Terlau et al., 1999). Chronically applied to whole oocytes or outside-out patches, kappa-PVIIA inhibition appears as a voltage-dependent relaxation in response to the depolarizing pulse used to activate the channels (Garcia et al., 1999).

Potassium channels are vital in controlling the resting membrane potential in excitable cells and can be broadly subdivided into three classes, voltage-gated channels, Ca²⁺ activated channels and ATP-sensitive K⁺ channels. ATP-sensitive potassium channels were originally described in cardiac tissue (Noma, 1983). In subsequent years they have also been identified in pancreatic cells, skeletal, vascular and neuronal tissue. This group of K⁺ channels are modulated by intracellular ATP levels and as such, couple cellular metabolism to electrical activity. Enhanced levels of ATP produce closure of the K_{ATP} channels. The K_{ATP} channel is thought to be an octomeric complex comprised of two different subunits in a 1:1 stoichiometry; a weakly inward rectifying K⁺ channel Kir6.x (6.1 or 6.2), which is thought to form the channel pore, and a sulphonylurea (SUR) subunit. So far, three variants of the SUR have been identified: SUR1, SUR2A and SUR2B. While the Kir6.2 subunit is common to K_{ATP} channels in cardiac, pancreatic and neuronal tissue (Kir6.1 is preferentially expressed in vascular smooth muscle tissue), the SUR is differentially expressed. Kir6.2/SUR1 reconstitute the neuronal/pancreatic beta-cell channel, whereas Kir6.2/SUR2A are proposed to reconstitute the cardiac K_{ATP} channels.

Potassium channels comprise a large and diverse group of proteins that, through maintenance of the cellular membrane potential, are fundamental in normal biological function. The potential therapeutic applications for compounds that open K⁺ channels are far-reaching and include treatments of a wide range of disease and injury states, including cerebral and cardiac ischemia and asthma. Recently, considerable interest has focused around the ability of K⁺ channel openers to produce relaxation of airway smooth muscle, and as such, these compounds may offer a novel approach to the treatment of bronchial asthma (Lin et al., 1998; Muller-Schweinitzer and Fozard, 1997; Morley, 1994; Barnes, 1992). Furthermore, the cardioprotective effects of K⁺ channel openers are now well established in experimental animal models of cardiac ischemia (Jung et al., 1998; Kouchi et al., 1998). Less is known about the ability of these compounds to limit neuronal damage caused from cerebral ischemia. Most progress in the treatment of cerebral ischemia has focused around the development of compounds to reduce the influx of sodium and calcium ions. K⁺ channel openers, which restore the resting membrane potential, could also be employed to reduce acute damage associated with an ischemic episode in neuronal tissue (Reshef et al., 1998; Wind et al., 1997), as well as reducing glutamate-induced excitotoxicity (Lauritzen et al., 1997). However, clinical use of K_{ATP} openers has been somewhat limited due to their cardiovascular side effects (i.e., drop in blood pressure).

Thus, it is desired to develop new agents for opening K_{ATP} channels which can be used to treat a wide range of disease and injury states, including cerebral and cardiac ischemia and asthma.

### SUMMARY OF THE INVENTION

The invention relates to uses of kappa-conotoxin PVIIA (κ-PVIIA), analogs and derivatives for activating ATP-sensitive K⁺ channels. The opening of ATP-sensitive K⁺ channels is useful for treating many physiological disorders as described in further detail herein.

More specifically, the present invention is directed to the use of κ-PVIIA, its analogs, derivatives and physiologically acceptable salts thereof for opening K_{ATP} channels which can be used to treat cardiac ischemia, neuronal ischemia, ocular ischemia and asthma.

For purposes of the present invention, κ-PVIIA refers to a peptide having the following general formula:
Cys-Xaa₁-Ile-Xaa₂-Asn-Gln-Xaa₃-Cys-Xaa₄-Gln-Xaa₅-Leu-Asp-Asp-Cys-Cys-Ser-Xaa₁-Xaa₃-Cys-Asn-Xaa₁-Xaa₄-Asn-Xaa₃-Cys-Val (SEQ ID NO:1), wherein Xaa₁ and Xaa₃ are independently Arg, homoarginine, ornithine, Lys, N-methyl-Lys, N,N-dimethyl-Lys, N,N,N-trimethyl-Lys, any synthetic basic amino acid, His or halo-His; Xaa₂ is Pro or hydroxy-Pro (Hyp); Xaa₄ is Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; and Xaa₅ is His or halo-His. The C-terminus may contain a free carboxyl group or an amide group. The halo is preferably bromine, chlorine or iodine. It is preferred that Xaa₁ is Arg and Xaa₅ is His. It is more preferred that Xaa₁ is Arg, Xaa₃ is Lys, Xaa₄ is Phe and Xaa₅ is His. It is further preferred that the C-terminus contains a free carboxyl group.

The κ-PVIIA analogs refer to peptides having the following formulas:

(SEQ ID NO:2)
κ-PVIIA[R18A]:
Cys-Arg-Ile-Hyp-Asn-Gln-Lys-Cys-Phe-Gln-His-Leu-Asp-Asp-Cys-
Cys-Ser-Ala-Lys-Cys-Asn-Arg-Phe-Asn-Lys-Cys-Val;
(SEQ ID NO:3)
κ-PVIIA[R22A]:
Cys-Arg-Ile-Hyp-Asn-Gln-Lys-Cys-Phe-Gln-His-Leu-Asp-Asp-Cys-
Cys-Ser-Arg-Lys-Cys-Asn-Ala-Phe-Asn-Lys-Cys-Val;
(SEQ ID NO:4)
κ-PVIIA[I3A]:
Cys-Arg-Ala-Hyp-Asn-Gln-Lys-Cys-Phe-GIn-His-Leu-Asp-Asp-Cys-
Cys-Ser-Arg-Lys-Cys-Asn-Arg-Phe-Asn-Lys-Cys-Val;
(SEQ ID NO:5)
κ-PVIIA[κ19A]:
Cys-Arg-Ile-Hyp-Asn-Gln-Lys-Cys-Phe-Gln-His-Leu-Asp-Asp-Cys-
Cys-Ser-Arg-Ala-Cys-Asn-Arg-Phe-Asn-Lys-Cys-Val;
(SEQ ID NO:6)
κ-PVIIA[R2A]:
Cys-Ala-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:7)
κ-PVIIA[F9A]:
Cys-Arg-Ile-Hyp-Asn-Gln-Lys-Cys-Ala-Gln-His-Leu-Asp-Asp-Cys-
Cys-Ser-Arg-Lys-Cys-Asn-Arg-Phe-Asn-Lys-Cys-Val;
(SEQ ID NO:8)
κ-PVIIA[κ25A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Ala-Cys-Val;
(SEQ ID NO:9)
κ-PVIIA[R2κ]:
Cys-Lys-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:10)
κ-PVIIA[κ7A]:
Cys-Arg-Ile-Hyp-Asn-Gln-Ala-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:11)
κ-PVIIA[F9M]:
Cys-Arg-Ile-Hyp-Asn-Gln-Lys-Cys-Met-Gln-His-Leu-Asp-Asp-Cys-
Cys-Ser-Arg-Lys-Cys-Asn-Arg-Phe-Asn-Lys-Cys-Val;
(SEQ ID NO:12)
κ-PVIIA[F9Y]:
Cys-Arg-Ile-Hyp-Asn-Gln-Lys-Cys-Tyr-Gln-His-Leu-Asp-Asp-Cys-
Cys-Ser-Arg-Lys-Cys-Asn-Arg-Phe-Asn-Lys-Cys-Val;
(SEQ ID NO:13)
κ-PVIIA[R2Q]:
Cys-Gln-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:14)
κ-PVIIA[H11A]:
Cys-Arg-Ile-Hyp-Asn-Gln-Lys-Cys-Phe-Gln-Ala-Leu-Asp-Asp-Cys-
Cys-Ser-Arg-Lys-Cys-Asn-Arg-Phe-Asn-Lys-Cys-Val;
(SEQ ID NO:15)
κ-PVIIA[D14A]:
Cys-Arg-Ile-Hyp-Asn-Gln-Lys-Cys-Phe-Gln-His-Leu-Asp-Ala-Cys-
Cys-Ser-Arg-Lys-Cys-Asn-Arg-Phe-Asn-Lys-Cys-Val;
(SEQ ID NO:16)
κ-PVIIA[Q6A]:
Cys-Arg-Ile-Hyp-Asn-Ala-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:17)
κ-PVIIA[N21A]:
Cys-Arg-Ile-Hyp-Asn-Gln-Lys-Cys-Phe-Gln-His-Leu-Asp-Asp-Cys-
Cys-Ser-Arg-Lys-Cys-Ala-Arg-Phe-Asn-Lys-Cys-Val;
(SEQ ID NO:18)
κ-PVIIA[S17A]:
Cys-Arg-Ile-Hyp-Asn-Gln-Lys-Cys-Phe-Gln-His-Leu-Asp-Asp-Cys-
Cys-Ala-Arg-Lys-Cys-Asn-Arg-Phe-Asn-Lys-Cys-Val;
(SEQ ID NO:19)
κ-PVIIA[N24A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Ala-Lys-Cys-Val;
(SEQ ID NO:20)
κ-PVIIA[L12A]:
Cys-Arg-Ile-Hyp-Asn-Gln-Lys-Cys-Phe-Gln-His-Ala-Asp-Asp-Cys-
Cys-Ser-Arg-Lys-Cys-Asn-Arg-Phe-Asn-Lys-Cys-Val;
(SEQ ID NO:21)
κ-PVIIA[D1A]:
Cys-Arg-Ile-Hyp-Asn-Gln-Lys-Cys-Phe-Gln-His-Leu-Ala-Asp-Cys-
Cys-Ser-Arg-Lys-Cys-Asn-Arg-Phe-Asn-Lys-Cys-Val;
(SEQ ID NO:22)
κ-PVIIA[Q10A]:
Cys-Arg-Ile-Hyp-Asn-Gln-Lys-Cys-Phe-Ala-His-Leu-Asp-Asp-Cys-
Cys-Ser-Arg-Lys-Cys-Asn-Arg-Phe-Asn-Lys-Cys-Val;
(SEQ ID NO:23)
κ-PVIIA[V27A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Ala;
(SEQ ID NO:24)
κ-PVIIA[O4A]:
Cys-Arg-Ile-Ala-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
and
(SEQ ID NO:25)
κ-PVIIA[N5A]:
Cys-Arg-Ile-Hyp-Ala-Gln-Lys-Cys-Phe-Gln-His-Leu-Asp-Asp-Cys-
Cys-Ser-Arg-Lys-Cys-Asn-Arg-Phe-Asn-Lys-Cys-Val.

It is preferred that the C-terminus contains a free carboxyl group.

The present invention further relates to derivatives of the above peptides in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; and the Asn, Ser, Thr or Hyp residues may be glycosylated (contain an N-glycan or an O-glycan). The Cys residues may be in D or L configuration and may optionally be substituted with homocysteine (D or L). The Tyr residues may also be substituted with the 3-hydroxyl or 2-hydroxylisomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The aliphatic amino acids may be substituted by synthetic derivatives bearing non-natural aliphatic branched or linear side chains CₙH₂ₙ₊₂ up to and including n=8.

Examples of synthetic aromatic amino acid include, but are not limited to, such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also http://www.amino-acids.com), incorporated herein by reference, by and available from RSP Amino Acid Analogues, Inc., Worcester, Mass. The residues containing protecting groups are deprotected using conventional techniques. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference.

In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Ser. No. 09/420,797, filed 19 Oct. 1999 and in PCT Application No. PCT/US99/24380, filed 19 Oct. 1999 (PCT Published Application No. WO 00/23092), each incorporated herein by reference. A preferred glycan is Gal(β1-3)GalNAc(α1-).

Optionally, in the above peptides, pairs of Cys residues may be replaced pairwise with isoteric lactam or ester-thioether replacements, such as Ser/(Glu or Asp), Lys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues.

### BRIEF DESCRIPTION OF THE FIGURES

FIG. 1 shows fluorimetry measurements of intracellular K⁺ (determined with PBFI dye) following exposure to increasing concentrations of κ-PVIIA in primary cultures of ventricular myocytes. The data shown is from one trial and is represented as mean change in fluorescence ±S.E.M. (*p<0.05, unpaired t-test).

FIGS. 2A-2B show fluorimetry measurements of membrane potential (determined with Di-8-ANEPPs dye) following exposure to increasing concentrations of κ-PVIIA in primary cultures of ventricular myocytes (FIG. 2A) or cortex (FIG. 2B). Cells were loaded into 96 well plates at least six days before the experiment. Results are expressed as Mean±SEM and represent average data from between two and five individual trials.

FIGS. 3A-3B are bar graphs showing the inhibition of the κ-PVIIA (100 nM) response with 10 nM Glibenclamide (Glib) in primary cultures of myocytes (FIG. 3A) or with 50 uM Tolbutamide (Tolb) in primary cultures of cortex (FIG. 3B). Data represents mean±S.E.M.

FIGS. 4A-4C are whole cell recordings showing currents elicited by κ-PVIIA in (FIG. 4A) cortical cells and (FIG. 4B) myocytes. FIG. 4C shows I-V relationship of κ-PVIIA-induced current from a cardiac myocyte.

FIG. 5 is a bar graph showing the protective effect of 10 nM κ-PVIIA against hypoxia induced depolarization. Bars represent Mean±S.E.M.

FIG. 6 shows the effect of increasing concentrations of κ-PVIIA on glutamate-induced (100 uM) excitotoxicity measured six hours following glutamate washout (three to six trials).

### DETAILED DESCRIPTION OF THE PREFERRED EMBODIMENT

The invention relates to uses of kappa-conotoxin PVIIA (κ-PVIIA), analogs and derivatives for activating ATP-sensitive K⁺ channels. The activation of ATP-sensitive K⁺ channels is useful for treating many physiological disorders, as described in further detail herein.

More specifically, the present invention is directed to the use of κ-PVIIA, analogs and derivatives for opening K_{ATP} channels which can be used to treat cardiac ischemia, neuronal ischemia, ocular ischemia and asthma.

The present invention, in another aspect, relates to a pharmaceutical composition comprising an effective amount of κ-PVIIA, analogs, derivatives or pharmaceutically acceptable salts. Such a pharmaceutical composition has the capability of acting as an activator for K_{ATP} channels. Thus, the pharmaceutical compositions of the present invention are useful in the treatment of the disorders noted above.

κ-PVIIA can be isolated from Conus purpurascens as described in U.S. Pat. No. 5,672,682, or it can be chemically synthesized by general synthetic methods such as described in U.S. Pat. No. 5,672,682. Alternatively, the native peptide can be synthesized by conventional recombinant DNA techniques (Sambrook et al., 1989) using the DNA encoding κ-PVIIA (Shon et al., 1998). The peptides are also synthesized using an automated synthesizer. Amino acids are sequentially coupled to an MBHA Rink resin (typically 100 mg of resin) beginning at the C-terminus using an Advanced Chem Tech 357 Automatic Peptide Synthesizer. Couplings are carried out using 1,3-diisopropylcarbodimide in N-methylpyrrolidinone (NMP) or by 2-(1H-benzotriazole-1-yl)-1,1,3,3-tetramethyluronium hexafluorophosphate (HBTU) and diethylisopropylethylamine (DIEA). The FMOC protecting group is removed by treatment with a 20% solution of piperidine in dimethylformamide (DMF). Resins are subsequently washed with DMF (twice), followed by methanol and NMP.

Muteins, analogs or active fragments, of the foregoing τ-conotoxin peptides are also contemplated here. See, e.g., Hammerland et al (1992). Derivative muteins, analogs or active fragments of the conotoxin peptides may be synthesized according to known techniques, including conservative amino acid substitutions, such as outlined in U.S. Pat. No. 5,545,723 (see particularly col. 2, line 50 to col. 3, line 8); U.S. Pat. No. 5,534,615 (see particularly col. 19, line 45 to col. 22, line 33); and U.S. Pat. No. 5,364,769 (see particularly col. 4, line 55 to col. 7, line 26), each incorporated herein by reference.

Pharmaceutical compositions containing a compound of the present invention or its pharmaceutically acceptable salts as the active ingredient can be prepared according to conventional pharmaceutical compounding techniques. See, for example, Remington's Pharmaceutical Sciences, 18th Ed. (1990, Mack Publishing Co., Easton, Pa.). Typically, a K_{ATP} channel activating amount of the active ingredient will be admixed with a pharmaceutically acceptable carrier. The carrier may take a wide variety of forms depending on the form of preparation desired for administration, e.g., intravenous, oral or parenteral. The compositions may further contain antioxidizing agents, stabilizing agents, preservatives and the like. For examples of delivery methods, see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable for passage through the gastrointestinal tract, while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

For example, administration of the active agent according to this invention may be achieved using any suitable delivery means, including:
(a) pump (see, e.g., Lauer & Hatton (1993), Zimm et al. (1984) and Ettinger et al. (1978));
(b), microencapsulation (see, e.g., U.S. Pat. Nos. 4,352,883; 4,353,888; and 5,084,350);
(c) continuous release polymer implants (see, e.g., U.S. Pat. No. 4,883,666);
(d) macroencapsulation (see, e.g., U.S. Pat. Nos. 5,284,761, 5,158,881, 4,976,859 and 4,968,733 and published PCT patent applications WO92/19195, WO 95/05452);
(e) naked or unencapsulated cell grafts to the CNS (see, e.g., U.S. Pat. Nos. 5,082,670 and 5,618,531);
(f) injection, either subcutaneously, intravenously, intra-arterially, intramuscularly, or to other suitable site; or
(g) oral administration, in capsule, liquid, tablet, pill, or prolonged release formulation.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in an therapeutically effective amount. By a “therapeutically effective amount” or simply “effective amount” of an active compound is meant a sufficient amount of the compound to treat or alleviate pain or to induce analgesia at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or spealists, and typically takes account of the disorder to be treated, the condition of the individual patient, the site of delivery, the method of administration and other factors known to practitioners. Examples of techniques and protocols can be found in Remington's Parmaceutical Sciences.

Dosage may be adjusted appropriately to achieve desired drug levels, locally or systemically. Typically, the active agents of the present invention exhibit their effect at a dosage range of from about 0.001 mg/kg to about 250 mg/kg, preferably from about 0.01 mg/kg to about 100 mg/kg, of the active ingredient and more preferably, from about 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the conotoxin peptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

Activators of K_{ATP} channels have therapeutic significance for the treatment of asthma, cardiac ischemia and cerebral ischemia, among others.

Asthma: Asthma is a serious and common condition that effects approximately 12 million people in the United States alone. This disorder is particularly serious in children and it has been estimated that the greatest number of asthma patients are those under the age of 18 (National Health Survey, National Center of Health Statistics, 1989). The disease is characterized by chronic inflammation and hyper-responsiveness of the airway which results in periodic attacks of wheezing and difficulty in breathing. An attack occurs when the airway smooth muscle become inflamed and swells as a result of exposure to a trigger substance. In severe cases, the airway may become blocked or obstructed as a result of the smooth muscle contraction. Further exacerbating the problem is the release of large quantities of mucus which also act to block the airway. Chronic asthmatics are most commonly treated prophylactically with inhaled corticosteroids and acutely with inhaled bronchodilators, usually b-2 agonists. However, chronic treatment with inhaled corticosteroids has an associated risk of immune system impairment, hypertension, osteoporosis, adrenal gland malfunction and an increased susceptibility to fungal infections (Rakel, 1997). In addition use of b-2 agonists has been reported in some cases to cause adverse reactions including tremor, tachycardia and palpitations and muscle cramps (Rakel, 1997). Therefore, there is great potential in developing anti-asthmatic agents with fewer side-effects.

K⁺ channel openers have been shown to be effective relaxants of airway smooth muscle reducing hyperactivity induced obstruction of intact airway. In cryopreserved human bronchi (Muller-Schweinitzer and Fozard, 1997) and in the isolated guinea pig tracheal preparation (Lin et al, 1998; Ando et al., 1997; Nielson-Kudsk, 1996; Nagai et al., 1991). K_{ATP} openers produced relaxation whether the muscle was contracted spontaneously or induced by a range of spasmogens. Under these conditions, the K⁺ channel openers are thought to be acting to produce a K⁺ ion efflux and consequent membrane hyperpolarization. As a result, voltage-sensitive Ca²⁺ channels would close and intracellular calcium levels would drop, producing muscular relaxation. The development of new and more specific K_{ATP} openers may offer a novel approach both to the prophylactic and symptomatic treatment of asthma.

K_{ATP} channels are present in many tissue types beyond just the target tissue, therefore their activation may result in unwanted side effects. In particular, as K_{ATP} channels are found in vascular smooth muscle, it is possible that in addition to the beneficial anti-asthmatic properties of K_{ATP} openers there could be an associated drop in blood pressure. It is possible that delivering the compound in inhalant form directly to the airway smooth muscle will allow the concentration of the compound to be reduced significantly thereby minimizing adverse reactions.

Cardiac Ischemia: While numerous subtypes of potassium channels in cardiac tissue have not yet been fully characterized, openers of K_{ATP} channels show great promise as cardioprotective agents. The beneficial vasodilatory effects afforded by K⁺ channel openers in patients with angina pectoris are now well established (Chen et al., 1997; Goldschmidt et al., 1996; Yamabe et al., 1995; Koike et al., 1995). Furthermore, the activation of K_{ATP} channels appears also to be involved in the acute preconditioning of the myocardium following brief ischemic periods, acting to reduce the risk (Pell et al., 1998) and size of the reperfusion infarct (Kouchi et al., 1998).

Direct evidence for the cytoprotective properties of K_{ATP} channels was demonstrated by Jovanovic et al. (1998a). In these studies, the DNA encoding for the Kir6.2/SUR2A (cardiac K_{ATP}) channel were transfected in COS-7 monkey cells and the degree of calcium loading monitored. Untransfected cells were demonstrated to be vulnerable to the increases in intracellular calcium seen following hypoxia/reoxygenation. However, the transfection of the cells with the K_{ATP} channel conferred resistance to the potentially damaging effects of the hypoxia-reoxygenation. Thus, the cardiac K_{ATP} channels are likely to play a significant role in protecting the myocardium against reperfusion injury.

Cerebral Ischemia: Although treatment of cerebral ischemia has advanced significantly over the past 30 years, cerebral ischemia (stroke) still remains the third leading cause of death in the United States. More than 500,000 new stroke/ischemia cases are reported each year. Even though initial mortality is high (38%), there are close to three million survivors of stroke in the United States, and yearly cost for rehabilitation of these patients in the United States is close to $17 billion (Rakel, 1997).

The initial cellular effects occur very rapidly (a matter of minutes) after an ischemic episode, whereas the actual cellular destruction does not occur until several hours or days following the infarction. Initial effects include depolarization due to bioenergetic failure, and inactivation of Na⁺ channels. Voltage-gated calcium channels are activated resulting in a massive rise in intracellular calcium. Further exacerbating the problem is a large transient release of glutamate which itself increases both Na⁺ and Ca²⁺ influx through ionotropic glutamate receptors. Glutamate also binds to metabotropic receptors, which results in activation of the inositol phosphate pathway. This sets off a cascade of intracellular events, including further release of calcium from intracellular stores. It is now well accepted that this initial overload of intracellular calcium ultimately leads to the delayed cytotoxicity that is seen hours or days later.

Recently it has been reported that dopaminergic neurons exposed to a very short hypoxic challenge will hyperpolarize primarily through an opening of K_{ATP} channels (Guatteo et al., 1998). This stimulatory effect was suggested to be a direct result of the increased metabolic demand and the consequent drop in intracellular ATP levels. Furthermore Jovanovic et al. (1998b) recently reported that cells transfected with DNA encoding for Kir6.2/SUR1 (neuronal K_{ATP}) channel showed increased resistance to injury caused through hypoxia-reoxygenation. Therefore, the opening of K_{ATP} channels may serve a vital cytoprotective role during short periods of reduced oxygen in neuronal tissue. Thus, there is great therapeutic potential in developing compounds that not only will act to prevent this calcium influx prophylactically, but will aid in reestablishing the normal resting membrane potential in damaged tissue. Treatment with κ-PVIIA will act to open K_{ATP} channels, inducing membrane hyperpolarization and indirectly producing closure of the voltage-gated Ca²⁺ channels, thereby preventing or reducing deleterious effects of a massive calcium influx.

In accordance with the present invention, it has been found that intravenous (IV) injection of concentrations of κ-PVIIA, far higher than those required to produce maximal hyperpolarization in tracheal cultures in vitro, had no effect on blood pressure or heart rate in the anesthetized rat.

Our preliminary data indicates that kappa-PVIIA induces glibenclamide-sensitive currents in primary cultures of myocytes in a highly potent manner. Furthermore, incubation of primary myocyte cultures in the presence of κ-PVIIA confers protection against hypoxia-induced depolarization.

### EXAMPLES

The present invention is described by reference to the following Examples, which are offered by way of illustration and are not intended to limit the invention in any manner. Standard techniques well known in the art or the techniques specifically described below were utilized.

### Example 1

### Experimental Methods

1. Cell Culture Protocol

Primary cultures of rat neonatal cortical cells, ventricular myocytes, tracheal smooth muscle cells and hippocampal cells were prepared. Cortical hemispheres were cleaned of meninges and the hippocampus removed and dissociated separately using 20 U/ml Papain. Cells were dissociated with constant mixing for 45 min at 37° C. Digestion was terminated with fraction V BSA (1.5 mg/ml) and Trypsin inhibitor (1.5 mg/ml) in 10 mls media (DMEM/F12±10% fetal Bovine serum ±B27 neuronal supplement; Life Technologies). Cells were gently triturated, to separate cells from surrounding connective tissue. Using a fluid-handling robot (Quadra 96, Tomtec) cells were settled onto Primaria-treated 96 well plates (Becton-Dickinson). Each well was loaded with approximately 25,000 cells. Plates were placed into a humidified 5% CO₂ incubator at 37° C. and kept for at least five days before fluorescence screening. Ventricles were diced into 2 mm square pieces and were digested in the presence of 20 U/ml Papain and trypsin/EDTA 1× (Life technologies). Smooth muscle cells on the surface of the trachea were cultured using the same digestive enzymes. Culturing techniques followed the method above.

2. Fluorimetry Assay

The saline solution used for the fluorimetric assay contained [in mM] 137 NaCl, 5 KCl, 10 HEPES, 25 Glucose, 3 CaCl₂, and 1 MgCl₂.

Di-8-ANEPPs: Voltage-sensitive dye. The effects of the compounds on membrane-potential were examined using the voltage-sensitive dye Di-8-ANEPPs. The Di-8-ANEPPs (2 uM) was dissolved in DMSO (final bath concentration 0.3%) and loaded into the cells in the presence of 10% pluronic acid. The plates were incubated for 40 min and then washed 4 times with the saline solution before starting the experiments. Di-8-ANEPPs crosses over the membrane in the presence of the pluronic acid creating a cytoplasmic pool of dye. Di-8-ANEPPs inserts into the plasma membrane where changes in potential result in molecular rearrangement. During hyperpolarization, the dye interchelates into the outer leaflet of the plasma membrane from the cytoplasmic reservoir of dye. Hyperpolarizations are represented as a positive shift and depolarizations as a negative shift in the fluorescence levels. ANEPs dyes show a fairly uniform 10% change in fluorescence intensity per 100 mV change in membrane potential and as such, fluorescence changes can be correlated to changes in membrane potential.

PBFI:K⁺ sensitive dye. A lipid-soluble AM ester of the PBFI dye was used to examine the effect of the κ-PVIIA on intracellular potassium levels. The dye was loaded into the cytoplasm with 20% pluronic acid where esterases cleave the dye from the ester effectively trapping the dye within the cell. Increases in intracellular potassium (K⁺i) are reflected as a rise in fluorescence and decreases in K⁺i as a drop in fluorescence. Cells were pre-incubated in 5 uM PBFI for three to four hours prior to screening. As with the Di-8-ANEPPs dye, the plates were rinsed four times with saline prior to beginning the experiments.

Fluo-3-Calcium-sensitive dye. To examine changes in intracellular calcium a lipid-soluble ester of the Fluo-3 dye (2 uM in DMSO. Final bath concentration of DMSO 0.3%) is loaded into the cells in the presence of 20% pluronic acid. The plates are incubated for 35 minutes and washed four times with saline solution before beginning the experiments. Increases and decreases in the concentration of intracellular calcium are reflected as positive and negative changes in the percent fluorescence respectively.

Ethidium homodimer-1: cellular viability dye. The degree of cellular damage produced by a cytotoxic agent was measured using the dye Ethidium homodimer-1 (Molecular probes). This dye will not cross intact plasma membranes, but is able to readily enter damaged cells. Upon binding nucleic acids, the dye undergoes a fluorescent enhancement. Thus, the degree of cellular damage can be correlated to the amount of fluorescence. In preparation for the excitotoxicity assay, the cells were rinsed three times and pretreated with the kappa-PVIIA or an equal volume of saline. The cells were incubated for 15 minutes and glutamate (5-500 uM) added to the appropriate lanes of the plate. The cells were incubated for a further 30 minutes, and washed thoroughly four times. The Ethidium Dye (4 uM) was loaded into all the wells and a reading was taken immediately. Readings were then taken at hourly intervals.

3. Fluorimetry Protocol

Fluorometric measurements are an averaging of cellular responses from approximately 25,000 cells per well of a 96 well plate. Cultures of cells from the cortex include at least pyramidal neurons, bipolar neurons, interneurons and astrocytes. Changes in membrane potential (Di-8-ANEPPs), cellular damage (Ethidium homodimer-1), intracellular K⁺ (PBFI) and Ca²⁺ (Fluo-3) were used as a measure of the response elicited with κ-PVIIA alone or with κ-PVIIA in the presence of specific receptor/ion channel agonists or antagonists. Concentration-responses were collected with the κ-PVIIA to determine the effective range. In order to minimize well-to-well variability, each well acted as its own control by comparing the degree of fluorescence in pretreatment to that in post-treatment. This normalization process allows comparison of relative responses from plate to plate and culture to culture. Mixed-cell populations in each well were measured with the fluorimeter and individual cell signaling responses were averaged. Statistics, including mean and standard error of the mean, from eight wells allowed for comparison of significant differences between treatments. Results were expressed as percent change in fluorescence. An initial reading of a plate was taken in saline solution. Measurements using the Di-8-ANEPPs, Fluo-3 or PBFI dyes were made at time intervals of 15 seconds, two minutes, five minutes, 10 minutes, 20 minutes and 30 minutes in the presence of the compound. Readings with Ethidium homodimer-1 were made at hourly intervals.

4. Tracheal Smooth Muscle Preparation

Guinea pigs were sacrificed by cervical dislocation and the trachea excised and cleaned of connective tissue. Trachea were cut into four or five sections and opened by cutting through the ring of cartilage opposite the tracheal muscle. Each segment was mounted in a organ bath containing (mM) NaCl 118.2; KCl 4.7; MgSO₄ 1.2; KH₂PO₄ 1.2; Glucose, 11.7; CaCl₂ 1.9 and NaHCO₃ 25.0. The bath was maintained at 37° C. and gassed with 95% O₂ and 5% CO₂. The preparation was maintained under 1 g of tension and equilibrated for 60 minutes before starting the experiment. Contractions were measured isometrically using a force-displacement transducer connected to a Grass polygraph. Following the 60 minutes equilibration period, the trachea were exposed to a submaximal concentration of histamine. This step was repeated until the contractile response to the spasmogen is consistent. The relaxant effects of increasing concentrations of kappa-PVIIA was determined in the absence and presence of the histamine.

5. Patch Clamp Recording

Whole-cell patch clamp recordings were made from cortical neurons on coverslips coated with Polyornithine/Poly-D-lysine (5 to 28 days in culture) and from myocytes on uncoated coverslips. Patch pipettes were pulled from thin-wall borosilicate glass and had resistances of 4M to 6M. Currents were recorded with an EPC 9 amplifier (HEKA) and controlled by software (Pulse, HEKA) run on a Macintosh power PC. Whole-cell currents were low-passed filtered at 10 kHz, digitized through a VR-10b digital data recorder to be stored on videotape at a sampling rate of 94 kHz. The intracellular pipette contained (in mM): 107 KCl, 33 KOH, 10 EGTA, 1 MgCl₂, 1 CaCl₂ and 10 HEPES. The solution was brought to pH 7.2 with NaOH and 0.1-0.5 mM Na₂ATP and 0.1 mM NaADP were added immediately before the experiment. The extracellular solution contained (in mM): 60 KCl, 80 NaCl, 1 MgCl₂, 0.1 CaCl₂ and 10 HEPES. The pH of the external solution was brought to pH 7.4 with NaOH. The high concentration of potassium results in a calculated reversal potential for potassium of −20 mV. As a result, if the holding potential is more negative than −20 mV, opening K channels will result in an inward flux of K ions and a downward deflection of the whole cell current. These solutions were chosen as the K_{ATP} channel has weak inward rectifying properties and as such, larger inward currents were anticipated. Experiments that are underway will address the effect of κ-PVIIA in solutions with low potassium levels.

6. Electrophysiology Solutions

Two extracellular solutions were used with different K⁺ ion and Na⁺ ion concentrations. Solution 1 contained 5 mM KCl and has a potassium equilibrium potential (Eₖ) of −84 mV, and solution 2 contained 60 mM and has a corresponding Ek of −20 mV. Extracellular solution 1 contained (in mM): 5 KCl, 135 NaCl, 1 MgCl₂, 0.1 CaCl₂ and 10 HEPES. The pH of the external solution was corrected to pH 7.4 with NaOH. Extracellular solution 2 contained (in mM): 60 KCl, 80 NaCl, 1 MgCl₂, 0.1 CaCl₂ and 10 HEPES. The pH of the external solution was corrected to pH 7.4 with NaOH. The intracellular pipette contained (in mM): 107 KCl, 33 KOH, 10 EGTA, 1 MgCl₂, 1 CaCl₂ and 10 HEPES. The solution was brought to pH 7.2 with NaOH and 0.1-0.5 mM Na₂ATP, and 0.1 mM NaADP was added immediately before the experiment.

7. Interpreting the Electrophysiology Results

In the presence of a low concentration of external K⁺ ions (solution 1) and at holding potentials more depolarized than −84 mV, the opening of K⁺ channels will result in an outward flux of K⁺ ions. In the presence of a high concentration of K⁺ (solution 2) the membrane potential would have to be more negative than −20 mV in order to see an outward movement of K ions. If the actual reversal potentials of the current evoked by κ-PVIIA in two different extracellular solutions are the same as the calculated values, it is highly likely that the κ-PVIIA-induced current is a result of the flux of K ions. The reversal potential of the current was calculated by holding the cell at the calculated Ek and running 500 ms voltage ramps from −100 mV to +80 mV both in the presence and absence of increasing concentrations of κ-PVIIA. The average of four control ramps was subtracted from the average of four ramps evoked in the presence of κ-PVIIA. The resultant trace was the actual current induced by the presence of the compound. This was fitted with a polynomial function and the reversal potential calculated.

8. Time-lapse Confocal Ca²⁺ Imaging

Cortical cell cultures were loaded with the fluorescent Ca²⁺ indicator Fluo3-AM (Molecular Probes, Eugene Oreg.; 2 mM final concentration with 0.1% Pluronic acid) 40 minutes prior to imaging experiments. Coverslips containing cells were mounted in a laminar flow perfusion chamber (Cornell-Bell design; Warner Instruments, Hamden, Conn.) and rinsed in saline (137 mM NaCl, 5 mM KCl, 3 mM CaCl₂, 1 mM MgCl₂, 10 mM HEPES, and 20 mM Sorbitol, pH 7.3) for at least five minutes to remove excess Fluo-3AM. Time-lapse images were collected on a Nikon PCM200 (Melville, N.Y.) confocal scanning laser microscope equipped with a Zeiss Axiovert 135 inverted microscope (Carl Zeiss, Inc., Thornwood, N.Y.) and downloaded with no frame averaging every 1.8 seconds to an optical memory disk recorder (Panasonic TQ3031F, Secaucus N.J.) (see methods further described in Kim et al., 1994). Image analysis were performed on a standardized 5×5 pixel area of cytoplasm in every astrocyte in the field to prevent bias in data analysis. Time course plots of intensity measurements (% change in fluorescence) were obtained using programs written by H. Sontheimer (Birmingham, Ala.) and plotted using Origin (MicroCal Northampton, Mass.). Routine analysis consisted of time course plots for up to 200 cells per field with at least five trials, thus yielding data analysis often from thousands of cells per experiment.

### Example 2

### Exposure to κ-PVIIA Produces a Dose-Dependent Decrease in Intracellular K⁺

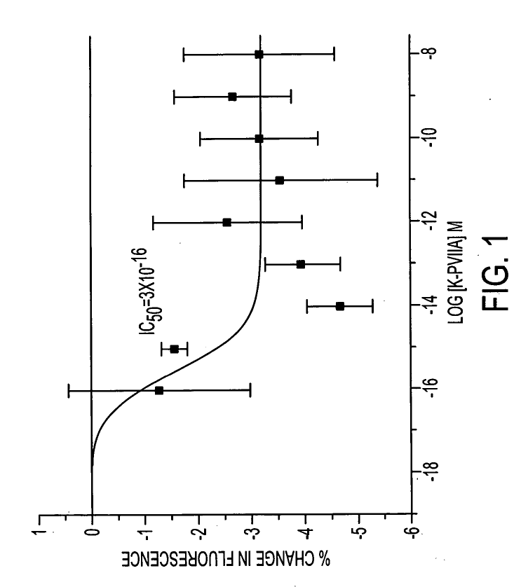

κ-PVIIA was originally isolated from the purple cone snail (Conus purpurascens) and was found to block the Drosophila H4 shaker K⁺ channel (Shon et al, 1998). In the same study no effects of the peptide were noted in oocytes expressing the mammalian shaker-like voltage-sensitive K⁺ channels Kv1.1 and Kv1.3. The potential of the peptide to block other voltage-gated K⁺ channels present in primary cultures of cortex was tested in this study. A 96-well fluorimetry assay was used to look for changes in potassium levels under depolarized conditions where voltage-gated potassium channels (Kv) would be activated. The cells were preloaded with the potassium indicator dye PBFI. If the compound acted to block Kv channels in a depolarized environment, there would be a resultant increase in intracellular K⁺. The results, however, suggested that at concentrations up to 100 nM, there was a reduction in the intracellular K⁺ concentration in untreated resting preparations (FIG. 1), as well as those preparations depolarized with 10-100 uM Aconitine. While the changes in fluorescence in the PBFI dye evoked with κ-PVIIA are small, it is important to stress that they are significant and repeatable.

### Example 3

### Exposure to κ-PVIIA Produces Dose-Dependent Hyperpolarization

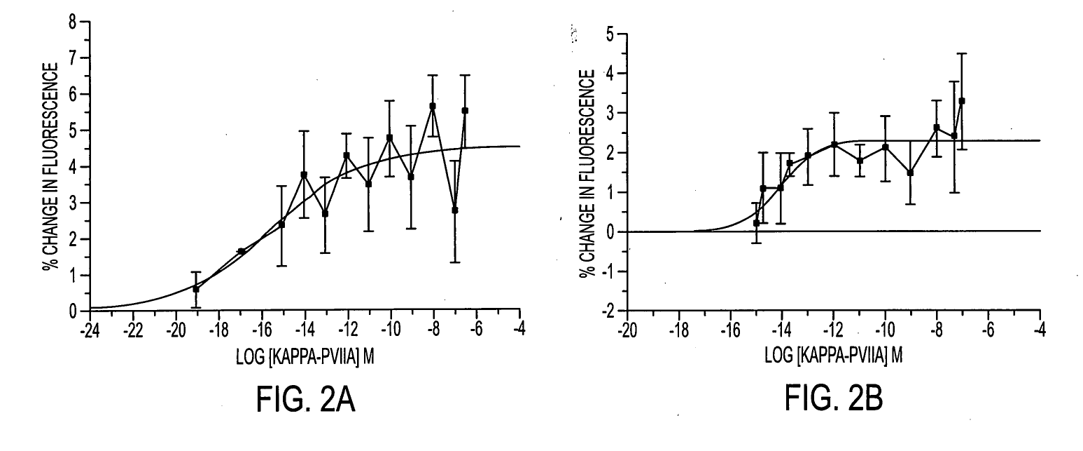

The fluorimetry experiments were repeated in the presence of the voltage-sensitive dye Di-8-ANEPPs, and the drop in intracellular K⁺ levels was seen to be accompanied by a significant hyperpolarization of the preparation (represented by a positive shift in the fluorescence, FIGS. 2A-2B). κ-PVIIA is extremely potent in this assay, showing EC₅₀s of 8×10⁻¹⁶ M in cortex, 9×10⁻¹⁶ M in myocyte cultures and 9×10⁻¹⁸ M in primary cultures of tracheal myocytes.

### Example 4

### The κ-PVIIA-Induced Hyperpolarization is Blocked by Exposure to K_{ATP} Antagonists

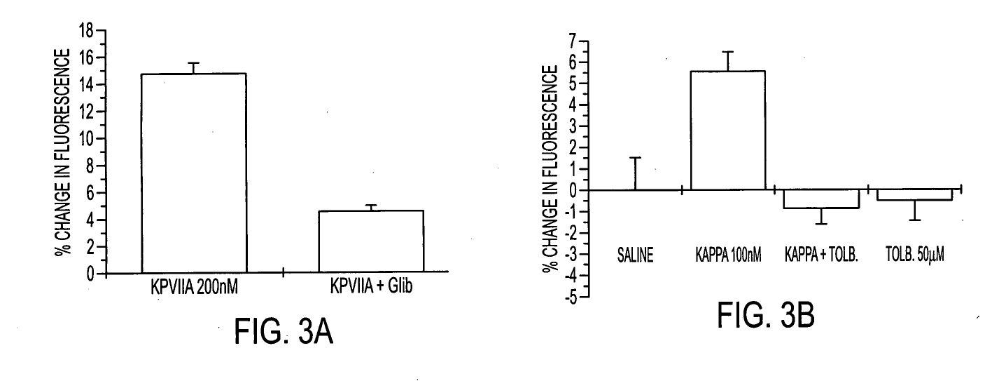

In order to determine the involvement of different K⁺ channel subtypes in the κ-PVIIA-induced hyperpolarization, effects of five well-documented K⁺ channel antagonists (4-aminopyridine (4-AP), Iberiotoxin (IBTX), Apamin, Tolbutamide and Glibenclamide) were tested. In cortical preparations, applications of 4-AP, IBTX and Apamin were without any detectable effect on the hyperpolarization seen with 100 nM κ-PVIIA. However, both Tolbutamide (1-10 uM) and Glibenclamide (10 nM), antagonists of the K_{ATP} channel, produced significant reductions in the κ-PVIIA induced hyperpolarization (FIG. 3B). Glibenclamide also produced significant reductions in the κ-PVIIA-induced hyperpolarization in cultures of myocytes (FIG. 3A).

### Example 5

### κ-PVIIA Induces Tolbutamide or Glibenclamide-Sensitive Currents

The sensitivity of the response to K_{ATP} antagonists was confirmed using the whole-cell patch clamp technique. In these experiments, the extracellular potassium concentration was increased to 60 mM and the solutions were calculated such that the reversal potential for potassium (Ek) would be −20 mV. Thus, the opening of K⁺ channels when the membrane potential is more negative than −20 mV will result in an influx of K⁺ ions. In both primary cultures of cortex and cardiac myocytes, the superfusion of 100 nM kappa-V11A induced an inward flux of positive ions that reversed close to −20 mV, indicating the involvement of K⁺ ions. With a holding potential of −80 mV, the currents evoked by κ-PVIIA were significantly larger in the myocyte preparation (87.7±5.9 pA, n=8) compared to the cortical preparation (26.2±6.2 pA, n=4). Even when the currents are corrected for cell capacitance, responses produced by the myocytes were greater than those seen in the cortical preparation (4.6±0.4 pA/pf and 2.4±0.7 pA/pf, respectively).

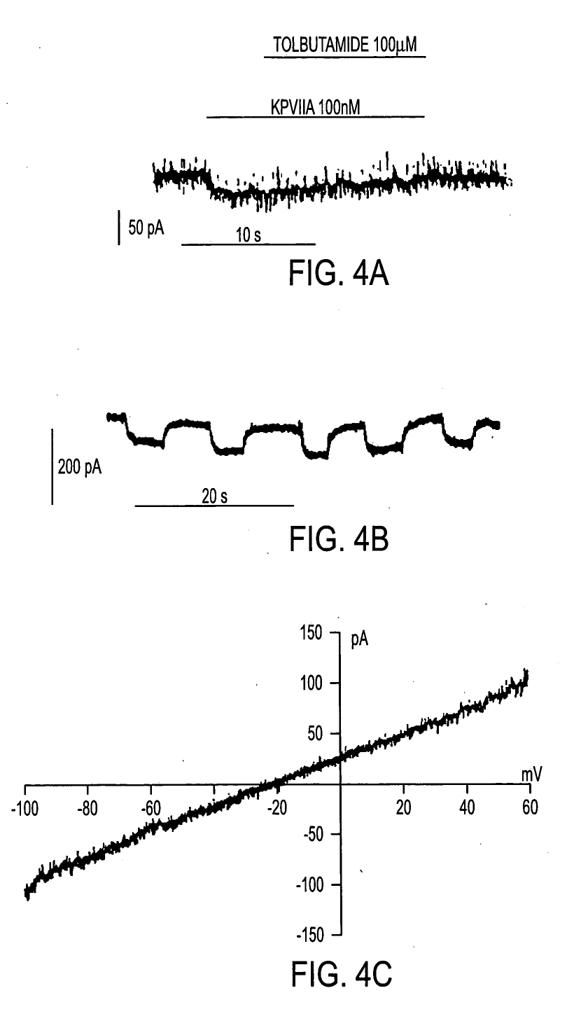

In both cases, the currents were sensitive either to the K_{ATP} antagonists tolbutamide (100 uM) or glibenclamide (10 nM) (FIGS. 4A and B). The reversal potential of the κ-PVIIA evoked current was determined using a voltage ramp from −100 to +60 mV and fitting the results with a fourth-order polynomial fit (FIG. 4C). The experimentally determined Ek (−23 mV) was close to the calculated Ek of −20 mV for these high potassium solutions, indicating the involvement of K⁺ channels.

### Example 6

### κ-PVIIA Produces a Slowly Developing Reduction in Intracellular Calcium

The effects of κ-PVIIA on intracellular calcium levels were determined using a 96-well fluorimetry assay plate and loading the cells with the Ca²⁺ indicator dye Fluo-3. In primary cultures of cortical neurons, κ-PVIIA produced a significant reduction in intracellular calcium. Little effect was noticeable with 1 nM κ-PVIIA at 15 seconds (−2.15±0.95%, two trials) but over time, the drop in calcium concentration became more profound (30 min, −8.8±3.9%).

### Example 7

### κ-PVIIA Protects Against Hypoxia-Induced Depolarization

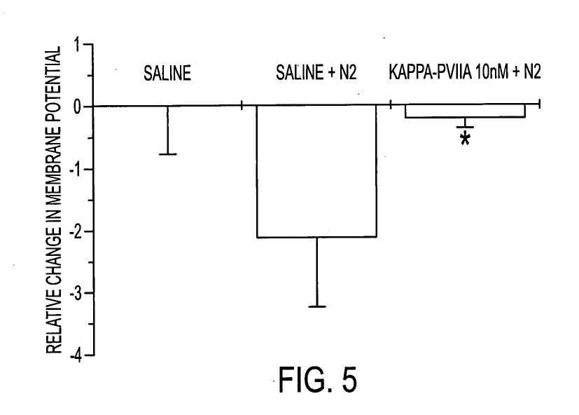

The depolarizing effects of N₂-induced hypoxia have been monitored in cardiac ventricular myocytes using the voltage sensitive dye Di-8-ANEPPs in a 96 well fluorimetry assay plate. Solutions were depleted of oxygen by constant bubbling with N₂ gas and were compared to results with control untreated saline. Under these conditions, hypoxia produced significant depolarization of the preparation (reflected as a drop in fluorescence), and incubating the preparation with 10 nM κ-PVIIA prevented any hypoxia-induced changes in membrane potential (FIG. 5).

### Example 8

### κ-PVIIA Protects Against Glutamate-Induced Excitotoxicity

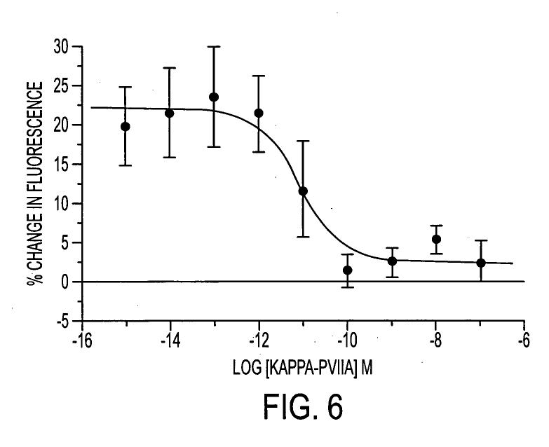

The protective effect of κ-PVIIA against glutamate-induced excitotoxicity was tested, using the 96-well fluorimetry assay and the Ethidium homodimer-1 dead cell dye. Five lanes of the 96-well plate were pre-exposed to 100 pM κ-PVIIA, and another five to control saline. Glutamate was then applied for 30 minutes, at which time the entire plate was washed thoroughly to remove all κ-PVIIA and glutamate. Ethidium dye was loaded, an initial reading taken and the amount of delayed cytotoxicity monitored for six hours. Increases in fluorescence represent increased cell destruction. As can be seen from FIG. 6, pre-incubating the cortical cells in κ-PVIIA resulted in very effective protection against the delayed (6 hrs) cytotoxic effects of 100 uM glutamate. This protection was blocked by 100 uM tolbutamide (K_{ATP} antagonist).

### Example 9

### Cytotoxicity of κ-PVIIA

Incubation of primary cortical cultures with 200 nM κ-PVIIA for 20 minutes induced no detectable protease activity (three trials). In comparison, a 20 minutes incubation with 5% Triton produced an ˜14% increase in fluorescence, as detected by the Enzchek protease-sensitive dye.

### Example 10

### Evaluation of κ-PVIIA as a Bronchodilator

The ability of κ-PVIIA to relax histamine-contracted, isolated Guinea pig tracheal segments is tested, using isometric tension recording. It is found that κ-PVIIA is able to relax histamine-contracted, isolated Guinea-pig tracheal segments. The response of κ-PVIIA is also tested in the presence of the K_{ATP} channel antagonists Tolbutamide or Glibenclamide. It is found that these antagonists reduce effects of κ-PVIIA, confirming involvement of the K_{ATP} channel in the response.

### Example 11

### Evaluating Protective Ability of κ-PVIIA in In Vitro Model of Hypoxia

A combination of the 96-well fluorimetric assay, electrophysiology, and confocal microscopy are used to assess the ability of κ-PVIIA to protect against the acute effects of transiently depleting oxygen in primary cultures. A multi-chamber saline reservoir has been constructed that allows the lower half of delivery plate to be filled with saline that is bubbled with N₂. Individual chambers allow the effects of decreasing oxygen to be monitored in the presence and absence of different concentrations of the κ-PVIIA. An initial screen in primary cultures of ventricular myocytes, using the potentiometric dye Di-8-ANEPPs, shows a strong protective effect of the κ-PVIIA against hypoxia induced depolarization. Similar effects are seen in the cortex and trachea. When the calcium-sensitive dye fluo-3 is used to observe changes in intracellular calcium levels induced by the hypoxic challenge, it is seen that κ-PVIIA is able to provide protection against hypoxia in all three tissue preparations. A similar result is obtained using the current-clamp mode of the whole cell patch clamp technique to monitor changes in membrane potential induced by hypoxia electrophysiology. This technique is very sensitive and allows the examination of the effect of κ-PVIIA on single tracheal, neuronal or myocyte cells.

### Example 12

### Evaluating Protective Ability of κ-PVIIA in In Vitro Model of Excitotoxicity

Preliminary fluorimetric experiments monitoring the degree of delayed cellular death produced following a challenge to a high concentration of glutamate have been carried out in primary cultures of cortex. The results indicate that the presence of the κ-PVIIA effectively reduces the degree of glutamate-induced excitotoxicity in a dose-dependant manner. Using the current-clamp mode of the whole-cell patch clamp technique, correlation of the fluorimetry results to actual changes in the membrane potential is examined. It is seen that the presence of the κ-PVIIA prevents the initial glutamate-induced depolarization, thereby conferring protection against the glutamate-induced calcium influx.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, described embodiments are illustrative and should not be construed as restrictive.

### LIST OF REFERENCES

Ando, T. et al. (1997). Clin. Exp. Allergy. 27:706-713.
Barnay, G. et al. (2000). J. Med. Chem.
Barnes, P. J. (1992). Eur. Respir. J 5:1126-1136.
Bitan, G. et al. (1997). J. Peptide Res. 49:421-426.
Chen, J. W. et al. (1997) Am. J. Cardiol. 80: 32-38.
Ettinger, L. J. et al. (1978). Cancer 41:1270-1273.
Garcia, E. et al. (1999). J. Gen. Physiol. 114:141-157.
Goldschmidt, M. et al. (1996). J. Clin. Pharmacol. 36:559-572.
Guatteo, E. et al. (1998). J. Neurophysiol. 79:1239-1245.
Hubry, V. et al. (1994). Reactive Polymers 22:231-241.
Jovanovic, A. et al. (1998a). Circulation 98:1548-1555.
Jovanovic, A. et al. (1998b). Lab. Invest. 78:1101-1107.
Jung, Y. S. et al. (1998). Japan J. Pharmacol. 76(1):65-73.
Kim et al. (1994).
Koike, A. et al. (1995). Am J. Cardiol. 76:449-452.
Kouchi, I. et al. (1998). Am. J. Physiol. 274(4/2); H1106-H1112.
Lauritzen I. et al. (1997). J. Neurochem. 69(4); 1570-1579.
Lin, C. et al. (1998). Pharmacology 57(6); 314-322.
Luer, M. S. & Hatton, J. (1993). Annals Pharmcotherapy 27:912-921.
Morley, J. (1994). Trends Pharmacol. Sci. 15(12); 463-468.
Muller-Shweinitzer, E. and Fozard, J. R. (1997). Br. J. Pharmacol. 120(7):1241-1248.
Nagai, H. et al. (1991). Japan J. Pharmacol. 56(1):13-21.
Neilson-Kudsk, J. E. (1996). Dan. Med. Bull. 43(5): 429-447.
Noma, A. (1983). Nature 304:147-148.
Ornstein, et al. (1993). Biorganic Medicinal Chemistry Letters 3:43-48.
Pell, T. J. et al. (1998). Am. J. Physiol. 275(5 pt 2):H542-547.
Rakel, R. E. (1997). Conn's current therapy.
Reshef A. et al. (1998). Neurosci. Lett. 250(2):111-114.
Sambrook, J. et al. (1989). Molecular Cloning: A Laboratory Manual, 2nd Ed., Cold Spring Harbor Laboratory, Cold Spring Harbor, N.Y.
Shon, K.-J. et al. (1998). J. Biol. Chem. 273:33-38.
Terlau, H. et al. (1996). Nature 381:148-151.
Terlau, H. et al. (1999). J. Gen. Physiol. 114:125-140.
Van de Steen, P. et al. (1998). Critical Rev. in Biochem. and Mol. Biol. 33:151-208.
Wind, T. et al. (1997). Brain Res. 751(2):295-299.
Yamabe H. et al. (1995). Cardiovasc. Drugs Ther. 9(6):755-761.
U.S. Pat. No. 5,331,001.
U.S. Pat. No. 5,534,615.
U.S. Pat. No. 5,364,769.
U.S. Pat. No. 5,545,723.
U.S. Pat. No. 5,550,050.
U.S. Pat. No. 5,844,077.
U.S. Pat. No. 5,672,682.
PCT Published Application WO 92/19195.
PCT Published Application WO 94/25503.
PCT Published Application WO 95/01203.
PCT Published Application WO 95/05452.
PCT Published Application WO 96/02286.
PCT Published Application WO 96/02646.
PCT Published Application WO 96/11698.
PCT Published Application WO 96/40871.
PCT Published Application WO 96/40959.
PCT Published Application WO 97/12635.
PCT Published Application WO 00/23092.

What is claimed is:

1. A method for treating disorders associated with radical depolarization of excitable membranes by activating a K_{ATP} channel which comprises administering to an individual in need thereof an effective amount of an active agent selected from the group consisting of:
(a) a compound of the following formula
Cys-Xaa₁-Ile-Xaa₂-Asn-Gln-Xaa₃-Cys-Xaa₄-Gln-Xaa₅-Leu-Asp-Asp-Cys-Cys-Ser-Xaa₁-Xaa₃-Cys-Asn-Xaa₁-Xaa₄-Asn-Xaa₃-Cys-Val (SEQ ID NO:1), wherein Xaa₁ and Xaa₃ are independently Arg, homoarginine, ornithine, Lys, N-methyl-Lys, N,N-dimethyl-Lys, N,N,N-trimethyl-Lys, any synthetic basic amino acid, His or halo-His; Xaa₂ is Pro or hydroxy-Pro (Hyp); Xaa₄ is Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; and Xaa₅ is His or halo-His,
(b) an analog of the compound of (a), said analog selected from the group consisting of:
(SEQ ID NO:2)
κ-PVIIA[R18A]:
Cys-Arg-Ile-Hyp-Asn-Gln-Lys-Cys-Phe-Gln-His-Leu-Asp-Asp-Cys-
Cys-Ser-Ala-Lys-Cys-Asn-Arg-Phe-Asn-Lys-Cys-Val;
(SEQ ID NO:3)
κ-PVIIA[R22A]:
Cys-Arg-Ile-Hyp-Asn-Gln-Lys-Cys-Phe-Gln-His-Leu-Asp-Asp-Cys-
Cys-Ser-Arg-Lys-Cys-Asn-Ala-Phe-Asn-Lys-Cys-Val;
(SEQ ID NO:4)
κ-PVIIA[I3A]:
Cys-Arg-Ala-Hyp-Asn-Gln-Lys-Cys-Phe-GIn-His-Leu-Asp-Asp-Cys-
Cys-Ser-Arg-Lys-Cys-Asn-Arg-Phe-Asn-Lys-Cys-Val;
(SEQ ID NO:5)
κ-PVIIA[κ19A]:
Cys-Arg-Ile-Hyp-Asn-Gln-Lys-Cys-Phe-Gln-His-Leu-Asp-Asp-Cys-
Cys-Ser-Arg-Ala-Cys-Asn-Arg-Phe-Asn-Lys-Cys-Val;
(SEQ ID NO:6)
κ-PVIIA[R2A]:
Cys-Ala-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:7)
κ-PVIIA[F9A]:
Cys-Arg-Ile-Hyp-Asn-Gln-Lys-Cys-Ala-Gln-His-Leu-Asp-Asp-Cys-
Cys-Ser-Arg-Lys-Cys-Asn-Arg-Phe-Asn-Lys-Cys-Val;
(SEQ ID NO:8)
κ-PVIIA[κ25A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Ala-Cys-Val;
(SEQ ID NO:9)
κ-PVIIA[R2κ]:
Cys-Lys-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:10)
κ-PVIIA[κ7A]:
Cys-Arg-Ile-Hyp-Asn-Gln-Ala-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:11)
κ-PVIIA[F9M]:
Cys-Arg-Ile-Hyp-Asn-Gln-Lys-Cys-Met-Gln-His-Leu-Asp-Asp-Cys-
Cys-Ser-Arg-Lys-Cys-Asn-Arg-Phe-Asn-Lys-Cys-Val;
(SEQ ID NO:12)
κ-PVIIA[F9Y]:
Cys-Arg-Ile-Hyp-Asn-Gln-Lys-Cys-Tyr-Gln-His-Leu-Asp-Asp-Cys-
Cys-Ser-Arg-Lys-Cys-Asn-Arg-Phe-Asn-Lys-Cys-Val;
(SEQ ID NO:13)
κ-PVIIA[R2Q]:
Cys-Gln-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:14)
κ-PVIIA[H11A]:
Cys-Arg-Ile-Hyp-Asn-Gln-Lys-Cys-Phe-Gln-Ala-Leu-Asp-Asp-Cys-
Cys-Ser-Arg-Lys-Cys-Asn-Arg-Phe-Asn-Lys-Cys-Val;
(SEQ ID NO:15)
κ-PVIIA[D14A]:
Cys-Arg-Ile-Hyp-Asn-Gln-Lys-Cys-Phe-Gln-His-Leu-Asp-Ala-Cys-
Cys-Ser-Arg-Lys-Cys-Asn-Arg-Phe-Asn-Lys-Cys-Val;
(SEQ ID NO:16)
κ-PVIIA[Q6A]:
Cys-Arg-Ile-Hyp-Asn-Ala-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:17)
κ-PVIIA[N21A]:
Cys-Arg-Ile-Hyp-Asn-Gln-Lys-Cys-Phe-Gln-His-Leu-Asp-Asp-Cys-
Cys-Ser-Arg-Lys-Cys-Ala-Arg-Phe-Asn-Lys-Cys-Val;
(SEQ ID NO:18)
κ-PVIIA[S17A]:
Cys-Arg-Ile-Hyp-Asn-Gln-Lys-Cys-Phe-Gln-His-Leu-Asp-Asp-Cys-
Cys-Ala-Arg-Lys-Cys-Asn-Arg-Phe-Asn-Lys-Cys-Val;
(SEQ ID NO:19)
κ-PVIIA[N24A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Ala-Lys-Cys-Val;
(SEQ ID NO:20)
κ-PVIIA[L12A]:
Cys-Arg-Ile-Hyp-Asn-Gln-Lys-Cys-Phe-Gln-His-Ala-Asp-Asp-Cys-
Cys-Ser-Arg-Lys-Cys-Asn-Arg-Phe-Asn-Lys-Cys-Val;
(SEQ ID NO:21)
κ-PVIIA[D1A]:
Cys-Arg-Ile-Hyp-Asn-Gln-Lys-Cys-Phe-Gln-His-Leu-Ala-Asp-Cys-
Cys-Ser-Arg-Lys-Cys-Asn-Arg-Phe-Asn-Lys-Cys-Val;
(SEQ ID NO:22)
κ-PVIIA[Q10A]:
Cys-Arg-Ile-Hyp-Asn-Gln-Lys-Cys-Phe-Ala-His-Leu-Asp-Asp-Cys-
Cys-Ser-Arg-Lys-Cys-Asn-Arg-Phe-Asn-Lys-Cys-Val;
(SEQ ID NO:23)
κ-PVIIA[V27A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Ala;
(SEQ ID NO:24)
κ-PVIIA[O4A]:
Cys-Arg-Ile-Ala-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
and
(SEQ ID NO:25)
κ-PVIIA[N5A]:
Cys-Arg-Ile-Hyp-Ala-Gln-Lys-Cys-Phe-Gln-His-Leu-Asp-Asp-Cys-
Cys-Ser-Arg-Lys-Cys-Asn-Arg-Phe-Asn-Lys-Cys-Val;
(c) a derivative of (a) or (b); and
(d) a physiologically acceptable salt thereof.

2. The method of claim 1, wherein Xaa₂ is hydroxy-Pro.

3. The method of claim 1, wherein Xaa₁ is Arg, Xaa₃ is Lys, Xaa₄ is Phe and Xaa₅ is His.

4. The method of claim 3, wherein Xaa₂ is hydroxy-Pro.

5. The method of claim 1, wherein said disorder is cardiac ischemia.

6. The method of claim 1, wherein said disorder is cerebral ischemia.

7. The method of claim 1, wherein said disorder is asthma.

8. The method of claim 1, wherein said disorder is ocular ischemia.

9. The method of claim 1, wherein the derivative is peptide of (a) or (b) in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; the Asn, Ser, Thr or Hyp residues may be glycosylated (contain an N-glycan or an O-glycan); the Cys residues may be in D or L configuration and may optionally be substituted with homocysteine (D or L); the Tyr residues may also be substituted with the 3-hydroxyl or 2-hydroxylisomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CₙH₂ₙ₊₂ up to and including n=8; and pairs of Cys residues may be replaced pairwise with isoteric lactam or ester-thioether replacements, such as Ser/(Glu or Asp), Lys/(Glu or Asp) or Cys/Ala combinations.

10. A method for treating cardiac ischemia which comprises administering to an individual in need thereof an effective amount of an active agent selected from the group consisting of:
(a) a compound of the following formula
Cys-Xaa₁-Ile-Xaa₂-Asn-Gln-Xaa₃-Cys-Xaa₄-Gln-Xaa₅-Leu-Asp-Asp-Cys-Cys-Ser-Xaa₁-Xaa₃-Cys-Asn-Xaa₁-Xaa₄-Asn-Xaa₃-Cys-Val (SEQ ID NO:1), wherein Xaa₁ and Xaa₃ are independently Arg, homoarginine, ornithine, Lys, N-methyl-Lys, N,N-dimethyl-Lys, N,N,N-trimethyl-Lys, any synthetic basic amino acid, His or halo-His; Xaa₂ is Pro or hydroxy-Pro (Hyp); Xaa₄ is Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; and Xaa₅ is His or halo-His,
(b) an analog of the compound of (a), said analog selected from the group consisting of:
(SEQ ID NO:2)
κ-PVIIA[R18A]:
Cys-Arg-Ile-Hyp-Asn-Gln-Lys-Cys-Phe-Gln-His-Leu-Asp-Asp-Cys-
Cys-Ser-Ala-Lys-Cys-Asn-Arg-Phe-Asn-Lys-Cys-Val;
(SEQ ID NO:3)
κ-PVIIA[R22A]:
Cys-Arg-Ile-Hyp-Asn-Gln-Lys-Cys-Phe-Gln-His-Leu-Asp-Asp-Cys-
Cys-Ser-Arg-Lys-Cys-Asn-Ala-Phe-Asn-Lys-Cys-Val;
(SEQ ID NO:4)
κ-PVIIA[I3A]:
Cys-Arg-Ala-Hyp-Asn-Gln-Lys-Cys-Phe-GIn-His-Leu-Asp-Asp-Cys-
Cys-Ser-Arg-Lys-Cys-Asn-Arg-Phe-Asn-Lys-Cys-Val;
(SEQ ID NO:5)
κ-PVIIA[κ19A]:
Cys-Arg-Ile-Hyp-Asn-Gln-Lys-Cys-Phe-Gln-His-Leu-Asp-Asp-Cys-
Cys-Ser-Arg-Ala-Cys-Asn-Arg-Phe-Asn-Lys-Cys-Val;
(SEQ ID NO:6)
κ-PVIIA[R2A]:
Cys-Ala-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:7)
κ-PVIIA[F9A]:
Cys-Arg-Ile-Hyp-Asn-Gln-Lys-Cys-Ala-Gln-His-Leu-Asp-Asp-Cys-
Cys-Ser-Arg-Lys-Cys-Asn-Arg-Phe-Asn-Lys-Cys-Val;
(SEQ ID NO:8)
κ-PVIIA[κ25A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Ala-Cys-Val;
(SEQ ID NO:9)
κ-PVIIA[R2κ]:
Cys-Lys-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:10)
κ-PVIIA[κ7A]:
Cys-Arg-Ile-Hyp-Asn-Gln-Ala-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:11)
κ-PVIIA[F9M]:
Cys-Arg-Ile-Hyp-Asn-Gln-Lys-Cys-Met-Gln-His-Leu-Asp-Asp-Cys-
Cys-Ser-Arg-Lys-Cys-Asn-Arg-Phe-Asn-Lys-Cys-Val;
(SEQ ID NO:12)
κ-PVIIA[F9Y]:
Cys-Arg-Ile-Hyp-Asn-Gln-Lys-Cys-Tyr-Gln-His-Leu-Asp-Asp-Cys-
Cys-Ser-Arg-Lys-Cys-Asn-Arg-Phe-Asn-Lys-Cys-Val;
(SEQ ID NO:13)
κ-PVIIA[R2Q]:
Cys-Gln-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:14)
κ-PVIIA[H11A]:
Cys-Arg-Ile-Hyp-Asn-Gln-Lys-Cys-Phe-Gln-Ala-Leu-Asp-Asp-Cys-
Cys-Ser-Arg-Lys-Cys-Asn-Arg-Phe-Asn-Lys-Cys-Val;
(SEQ ID NO:15)
κ-PVIIA[D14A]:
Cys-Arg-Ile-Hyp-Asn-Gln-Lys-Cys-Phe-Gln-His-Leu-Asp-Ala-Cys-
Cys-Ser-Arg-Lys-Cys-Asn-Arg-Phe-Asn-Lys-Cys-Val;
(SEQ ID NO:16)
κ-PVIIA[Q6A]:
Cys-Arg-Ile-Hyp-Asn-Ala-Lys-Cys-Phe-Gln-His-Leu-Asp-Asp-Cys-
Cys-Ser-Arg-Lys-Cys-Asn-Arg-Phe-Asn-Lys-Cys-Val;
(SEQ ID NO:17)
κ-PVIIA[N21A]:
Cys-Arg-Ile-Hyp-Asn-Gln-Lys-Cys-Phe-Gln-His-Leu-Asp-Asp-Cys-
Cys-Ser-Arg-Lys-Cys-Ala-Arg-Phe-Asn-Lys-Cys-Val;
(SEQ ID NO:18)
κ-PVIIA[S17A]:
Cys-Arg-Ile-Hyp-Asn-Gln-Lys-Cys-Phe-Gln-His-Leu-Asp-Asp-Cys-
Cys-Ala-Arg-Lys-Cys-Asn-Arg-Phe-Asn-Lys-Cys-Val;
(SEQ ID NO:19)
κ-PVIIA[N24A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Ala-Lys-Cys-Val;
(SEQ ID NO:20)
κ-PVIIA[L12A]:
Cys-Arg-Ile-Hyp-Asn-Gln-Lys-Cys-Phe-Gln-His-Ala-Asp-Asp-Cys-
Cys-Ser-Arg-Lys-Cys-Asn-Arg-Phe-Asn-Lys-Cys-Val;
(SEQ ID NO:21)
κ-PVIIA[D1A]:
Cys-Arg-Ile-Hyp-Asn-Gln-Lys-Cys-Phe-Gln-His-Leu-Ala-Asp-Cys-
Cys-Ser-Arg-Lys-Cys-Asn-Arg-Phe-Asn-Lys-Cys-Val;
(SEQ ID NO:22)
κ-PVIIA[Q10A]:
Cys-Arg-Ile-Hyp-Asn-Gln-Lys-Cys-Phe-Ala-His-Leu-Asp-Asp-Cys-
Cys-Ser-Arg-Lys-Cys-Asn-Arg-Phe-Asn-Lys-Cys-Val;
(SEQ ID NO:23)
κ-PVIIA[V27A]:
Cys-Arg-Ile-Hyp-Asn-Gln-Lys-Cys-Phe-Gln-His-Leu-Asp-Asp-Cys-
Cys-Ser-Arg-Lys-Cys-Asn-Arg-Phe-Asn-Lys-Cys-Ala;
(SEQ ID NO:24)
κ-PVIIA[O4A]:
Cys-Arg-Ile-Ala-Asn-Gln-Lys-Cys-Phe-Gln-His-Leu-Asp-Asp-Cys-
Cys-Ser-Arg-Lys-Cys-Asn-Arg-Phe-Asn-Lys-Cys-Val;
and
(SEQ ID NO:25)
κ-PVIIA[N5A]:
Cys-Arg-Ile-Hyp-Ala-Gln-Lys-Cys-Phe-Gln-His-Leu-Asp-Asp-Cys-
Cys-Ser-Arg-Lys-Cys-Asn-Arg-Phe-Asn-Lys-Cys-Val;
(c) a derivative of (a) or (b); and
(d) a physiologically acceptable salt thereof.

11. The method of claim 10, wherein the size of reperfusion infarct resulting from cardiac ischemia is reduced.

12. The method of claim 10, wherein Xaa₂ is hydroxy-Pro.

13. The method of claim 12, wherein the size of reperfusion infarct resulting from cardiac ischemia is reduced.

14. The method of claim 10, wherein Xaa₁ is Arg, Xaa₃ is Lys, Xaa₄ is Phe and Xaa₅ is His.

15. The method of claim 14, wherein the size of reperfusion infarct resulting from cardiac ischemia is reduced.

16. The method of claim 14, wherein Xaa₂ is hydroxy-Pro.

17. The method of claim 16, wherein the size of reperfusion infarct resulting from cardiac ischemia is reduced.

18. The method of claim 10, wherein the derivative is peptide of (a) or (b) in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; the Asn, Ser, Thr or Hyp residues may be glycosylated (contain an N-glycan or an O-glycan); the Cys residues may be in D or L configuration and may optionally be substituted with homocysteine (D or L); the Tyr residues may also be substituted with the 3-hydroxyl or 2-hydroxylisomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CₙH₂ₙ₊₂ up to and including n=8; and pairs of Cys residues may be replaced pairwise with isoteric lactam or ester-thioether replacements, such as Ser/(Glu or Asp), Lys/(Glu or Asp) or Cys/Ala combinations.

19. The method of claim 18, wherein the size of reperfusion infarct resulting from cardiac ischemia is reduced.

20. The method of claim 1, wherein the active agent is a compound of the formula Cys-Arg-Ile-Xaa-Asn-Gln-Lys-Cys-Phe-Gln-His-Leu-Asp-Asp-Cys-Cys-Ser-Arg-Lys-Cys-Asn-Arg-Phe-Asn-Lys-Cys-Val (SEQ ID NO:26), wherein Xaa is hydroxy-Pro.

21. The method of claim 10, wherein the active agent is a compound of the formula Cys-Arg-Ile-Xaa-Asn-Gln-Lys-Cys-Phe-Gln-His-Leu-Asp-Asp-Cys-Cys-Ser-Arg-Lys-Cys-Asn-Arg-Phe-Asn-Lys-Cys-Val (SEQ ID NO:26), wherein Xaa is hydroxy-Pro.


SEQUENCE LISTING (26 sequences)

SEQ ID NO: 1 - protein, 27 aa, Conus purpurascens
  PEPTIDE (1)..(27): Xaa at residue 2, 7, 18, 19, 22 and 25 may be Arg, homoarginine, ornithine, Lys, N-methyl-Lys, N,N-dimethyl-Lys, N,N,N-trimethyl-Lys, any synthetic basic amino acid, His or halo-His; Xaa at
CXIXNQXCXQXLDDCCSXXCNXXNXCV

SEQ ID NO: 2 - protein, 27 aa, Conus purpurascens
  PEPTIDE (1)..(27): Xaa is Hyp
CRIXNQKCFQHLDDCCSAKCNRFNKCV

SEQ ID NO: 3 - protein, 27 aa, Conus purpurascens
  PEPTIDE (1)..(27): Xaa is Hyp
CRIXNQKCFQHLDDCCSRKCNAFNKCV

SEQ ID NO: 4 - protein, 27 aa, Conus purpurascens
  PEPTIDE (1)..(27): Xaa is Hyp
CRAXNQKCFQHLDDCCSRKCNRFNKCV

SEQ ID NO: 5 - protein, 27 aa, Conus purpurascens
  PEPTIDE (1)..(27): Xaa is Hyp
CRIXNQKCFQHLDDCCSRACNRFNKCV

SEQ ID NO: 6 - protein, 27 aa, Conus purpurascens
  PEPTIDE (1)..(27): Xaa is Hyp
CAIXNQKCFQHLDDCCSRKCNRFNKCV

SEQ ID NO: 7 - protein, 27 aa, Conus purpurascens
  PEPTIDE (1)..(27): Xaa is Hyp
CRIXNQKCAQHLDDCCSRKCNRFNKCV

SEQ ID NO: 8 - protein, 27 aa, Conus purpurascens
  PEPTIDE (1)..(27): Xaa is Hyp
CRIXNQKCFQHLDDCCSRKCNRFNACV

SEQ ID NO: 9 - protein, 27 aa, Conus purpurascens
  PEPTIDE (1)..(27): Xaa is Hyp
CKIXNQKCFQHLDDCCSRKCNRFNKCV

SEQ ID NO: 10 - protein, 27 aa, Conus purpurascens
  PEPTIDE (1)..(27): Xaa is Hyp
CRIXNQACFQHLDDCCSRKCNRFNKCV

SEQ ID NO: 11 - protein, 27 aa, Conus purpurascens
  PEPTIDE (1)..(27): Xaa is Hyp
CRIXNQKCMQHLDDCCSRKCNRFNKCV

SEQ ID NO: 12 - protein, 27 aa, Conus purpurascens
  PEPTIDE (1)..(27): Xaa is Hyp
CRIXNQKCYQHLDDCCSRKCNRFNKCV

SEQ ID NO: 13 - protein, 27 aa, Conus purpurascens
  PEPTIDE (1)..(27): Xaa is Hyp
CQIXNQKCFQHLDDCCSRKCNRFNKCV

SEQ ID NO: 14 - protein, 27 aa, Conus purpurascens
  PEPTIDE (1)..(27): Xaa is Hyp
CRIXNQKCFQALDDCCSRKCNRFNKCV

SEQ ID NO: 15 - protein, 27 aa, Conus purpurascens
  PEPTIDE (1)..(27): Xaa is Hyp
CRIXNQKCFQHLDACCSRKCNRFNKCV

SEQ ID NO: 16 - protein, 27 aa, Conus purpurascens
  PEPTIDE (1)..(27): Xaa is Hyp
CRIXNAKCFQHLDDCCSRKCNRFNKCV

SEQ ID NO: 17 - protein, 27 aa, Conus purpurascens
  PEPTIDE (1)..(27): Xaa is Hyp
CRIXNQKCFQHLDDCCSRKCARFNKCV

SEQ ID NO: 18 - protein, 27 aa, Conus purpurascens
  PEPTIDE (1)..(27): Xaa is Hyp
CRIXNQKCFQHLDDCCARKCNRFNKCV

SEQ ID NO: 19 - protein, 27 aa, Conus purpurascens
  PEPTIDE (1)..(27): Xaa is Hyp
CRIXNQKCFQHLDDCCSRKCNRFAKCV

SEQ ID NO: 20 - protein, 27 aa, Conus purpurascens
  PEPTIDE (1)..(27): Xaa is Hyp
CRIXNQKCFQHADDCCSRKCNRFNKCV

SEQ ID NO: 21 - protein, 27 aa, Conus purpurascens
  PEPTIDE (1)..(27): Xaa is Hyp
CRIXNQKCFQHLADCCSRKCNRFNKCV

SEQ ID NO: 22 - protein, 27 aa, Conus purpurascens
  PEPTIDE (1)..(27): Xaa is Hyp
CRIXNQKCFAHLDDCCSRKCNRFNKCV

SEQ ID NO: 23 - protein, 27 aa, Conus purpurascens
  PEPTIDE (1)..(27): Xaa is Hyp
CRIXNQKCFQHLDDCCSRKCNRFNKCA

SEQ ID NO: 24 - protein, 27 aa, Conus purpurascens
CRIANQKCFQHLDDCCSRKCNRFNKCV

SEQ ID NO: 25 - protein, 27 aa, Conus purpurascens
  PEPTIDE (1)..(27): Xaa is Hyp
CRIXAQKCFQHLDDCCSRKCNRFNKCV

SEQ ID NO: 26 - protein, 27 aa, Conus purpurascens
  PEPTIDE (1)..(27): Xaa is Hyp
CRIXNQKCFQHLDDCCSRKCNRFNKCV